(19) United States
(12) Patent Application Publication
(10) Pub. No.: US 2005/0276855 A1
(43) Pub. Date: Dec. 15, 2005

(54) Composition and method using local application of lipophilic lathyrogens sustained release formulations

(76) Inventor: Milos Chvapil, Tucson, AZ (US)

Correspondence Address: DALE F. REGELMAN, LAW OFFICE OF DALE F. REGELMAN, P.C., 4231 SOUTH FREMONT AVENUE, TUCSON, AZ, 85714, US

(21) Appl. No.: 11/153,582

(22) Filed: Jun. 15, 2005

Related U.S. Application Data
(60) Provisional application No. 60/579,772, filed on Jun. 15, 2004.

(51) Int. Cl. A61K 9/14; A61K 31/22

(52) U.S. Cl. 424/486; 514/562; 514/550

(57) ABSTRACT

A method is disclosed to treat a patient having a wound in a tissue structure, where the wound is defined by a wound line, by inhibiting the crosslinking of collagen during the healing of that wound. The method provides a lipophilic lathyrogen, and injects that lipophilic lathyrogen into the tissue structure adjacent the wound line. The wound may result from either injury or surgery. The would may comprise a skin wound, a stenoses, a stricture, a burn, a fibrotic adhesion, and the like.

### CROSS REFERENCE TO RELATED APPLICATIONS

This application claims priority from a U.S. Provisional Application having Ser. No. 60/579,772.

### FIELD OF THE INVENTION

The invention relates to a composition and method using local application of lipophilic lathyrogens in sustained release formulations. In certain embodiments, the invention is directed to inhibiting the crosslinking of collagen during wound healing.

### BACKGROUND OF THE INVENTION

Any deep wound created either by injury or by surgical intervention heals by a sequence of events resulting in the formation of a fibrotic scar. In this process the starting cellular-humoral phase is followed by the accumulation first of glycosaminoglycans (GAG) and then by the synthesis and deposition of collagen. At this initial stage of the healing process, the collagen molecules are only aggregated, i.e. holding together by weak linkages which are easily dissociated by saline or by weak acids, and which do not provide the collagen with mechanical strength.

In the following stage of the healing process the still increased population of fibroblast in the repair tissue starts synthesizing a lysyl oxidase, which is the enzyme initiating the formation of strong covalent intra- but most importantly intermolecular crosslinks. The aggregated, non-crosslinked, collagen is loosely packed and binds large volumes of fluids. The crosslinked molecules are, however, tightly packed and their water binding capacity is much reduced. As a consequence the mass of collagen, and the whole scar tissue, shrinks forming the pathologies such as stenosis, stricture, contracture, strong fibrotic adhesions.

### SUMMARY OF THE INVENTION

Applicant's invention comprises a method to treat a patient having a wound in a tissue structure, wherein the wound is defined by a wound line, by inhibiting the crosslinking of collagen during the healing of that wound. Applicant's method provides a lipophilic lathyrogen, and injects that lipophilic lathyrogen into the tissue structure adjacent the wound line. The wound may result from either injury or surgery. The would may comprise a skin wound, a stenoses, a stricture, a burn, a fibrotic adhesion, and the like.

Applicant's invention further comprises a sustained release composition comprising one or more lipophilic lathyrogens. In certain embodiments, Applicant's sustained release composition may further comprise a polymeric carrier.

### DETAILED DESCRIPTION OF THE PREFERRED EMBODIMENTS

The use of local applications of lipophilic lathyrogens in clinical situations will be described in the following sections.

1. Clinical Applications

1.1 Stenoses 1.1.1. carotis restenosis
   1.1.2. stenosis of esophagus
   1.1.3. stenosis of trachea
1.2. Strictures 1.2.1. urethral stricture
   1.2.2. trabeculectomy in glaucoma surgery
1.3 Joint stiffness
   1.3.1 immobilized joints
   1.3.2 temporomandibular joint
1.4 Scar contracture
   1.4.1 in 3^{rd} degree burns
   1.4.2 in expansion presbyopia surgery
   1.4.3 in glaucoma filtration surgery
1.5 Fibrotic adhesions
   1.5.1 peritendinous
   1.5.2 perineural
   1.5.3 peritoneal
1.6 Potential candidates for lipophilic lathyrogens local treatment
   1.6.1 Dupuytran contracture of palmar aponeurosis
   1.6.2 Adhesions or stricture of Fallopian tube
   1.6.3 Carpal tunnel syndrom
   1.6.4 Intestinal strictures (Crohn disease)

It has been recognized that is not the amount of newly deposited collagen in a tissue or organ which creates the pathology. Rather, collagen crosslinking turns the “soft” collagen into a rigid, mechanically strong structure resulting in the above listed pathologies.

Collagen crosslinking is related to the synthesis and function of the enzyme lysyl oxidase (L.O.). This event takes place later in the healing process. The actual time of the accumulation and function of lysyl oxidase in the repair tissue depends on various factors, such as type of affected tissue, species, age, sex, and physical and nutritional factors. Basic studies on the healing dynamics of various tissues in various species established with reasonable accuracy the time of lysyl oxidase activity, which last only for a certain time and declines with the reduction of fibroblasts presence and their activity. Once the collagen crosslinks are established, the enzyme is no longer synthesized and active.

Lysyl oxidase oxidatively desaminates some residues of LYS and HYL, forming aldehydes. These then non-enzymatically form the actual covalent inter- and intra-molecular crosslinks by reaction between each other, i.e. an aldol condensation, or reaction with free —NH₂ group disposed on adjacent LYS or HYL moieties.

This second type of crosslinking forms Schiff base covalent crosslinks, which with time, albeit very slowly, revert into stable reduced crosslinks. The non-reduced crosslinks remain labile and susceptible to cleavage by heat and acids. The reduced crosslinks are resistant to these factors. Both crosslinks, however, contribute to the mechanical strength of the collagen fibers—under physiologic conditions. At temperatures above 40° C. and in acid environment of pH less than 4.2, the Schiff base crosslinks are cleaved and the collagen structure looses its stability and strength. This is the suggested mechanism of the partial rupture of the superficial digital flexor tendon in racing horses, where both increased local temperature and the acidity was reported.

Beta-aminopropionitrile (“BAPN”), i.e. Compound VII, and D-Penicillamine (“DPA”), i.e. Compound VI, inhibit lysyl oxidase activity. 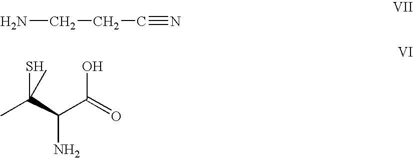
Applicant has found that BAPN and/or DPA should be administered temporarily during the synthesis and function of lysyl oxidase. As a general matter, BAPN and/or DPA should be administered 10-15 days after infliction of the injury, and that administration should last for about 4-6 weeks which is the general time of L.O. synthesis and activity in the wound tissue. In later healing stages the activity of the enzyme is very much reduced. These time frames assume that the healing process is completed within about 2 months.

Although BAPN and/or DPA specifically block only the formation of collagen crosslinks and not synthesis of this macromolecule, these drugs increases the pool of soluble collagens in the repair tissue by disintegrating the original collagen fibrilar structure to smaller fragments. As tissue collagenases degrade much faster non-crosslinked collagen, it follows that collagen catabolism exceeds collagen synthesis and deposition. By this mechanism both lathyrogens reduce the mass of deposited collagen, as has been reported.

Two findings justify a continuous delivery of these drugs into the site of the fibrotic lesion. It is known that lysyl oxidase, once inactivated by BAPN, is quickly within 24 hours resynthesized. It is further known that BAPN and DPA are metabolized rather quickly to inactive products.

A continuous, daily systemic administration of either drug at effective dose to interfere with the activity of L.O. is marred by development of several side toxic effects, which often require discontinuation of the treatment. This inventor recognized the need for topical or local administration of lathyrogens in the treatment of fibrotic lesions at effective dose many time smaller than that used in systemic treatment. Thus, no side toxic effects were present. This is object of U.S. Pat. No. 4,485,088: Method of treatment of fibrotic lesions by topical administration of lathyrogenic drugs. Still this patent requires daily local or topical administration of either drug for reasons indicated above.

This invention progresses these ideas by developing a method wherein the hydrophilic drugs are converted by chemical methods to lipophilic moiety, allowing a slow release of the drug as such or mixed into a polymer delivery system from the tissue depot.

In the next part the individual pathologies, as listed above are briefly discussed to document that it has been already shown in animal models and in few human trials that a systemic administration of either drug was effective to reduce the gravity of the disease, in spite of often manifestation of side toxic effects.

### 1.1. Stenosis

### 1.1.1. Coronary Artery Restenosis

Heart disease, the most common cause of death in North America and Europe, eventually manifests itself as heart muscle ischemia, i.e. angina and/or myocardial infarction. After diagnostic angiography locates the site of coronary artery narrowing, the area is most often treated with balloon, laser or artherectomy luminal diameter correction (angioplasty) with or without insertion of luminal stabilizing stent.

In spite of these sophisticated maneuvers, coronary artery restenosis occurs in 35-45% of unstented and 20 to 30% of stented lesions, usually within 6-12 months post treatment. Failure of this therapy results in coronary artery bypass surgery with its own incidence of restenosis and occlusion. To date, research efforts to understand and combat the fibromuscular neointimal hyperplasia and new scar proliferation associated with restenosis has failed to respond to the application of localized anti-tumor, anti-cellular and radiation therapies.

Several studies have documented that in early stages after angioplasty the narrowing of the artery lumen is related to thrombus formation and hyperplasia of smooth muscle cells. Months after the angioplasty, it is the collagen accumulation and maturation which causes the stenosis. It is a classic case of fibroproductive inflammation, as described above.

Post angioplasty new scar formation with collagen deposition and in later stages followed by crosslinking of collagen with stenosis, can be pharmacologically altered to allow permanent luminal remodeling of angioplastied arteries under the effect of long acting BAPN (IV) or DPA (II).

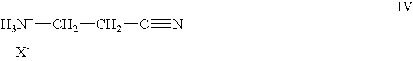

Spears et al. (1994) studied the effect of BAPN, administered systemically to rabbits having the vessel luminal narrowing induced either by conventional balloon angioplasty or by laser balloon angioplasty. They concluded that BAPN may be efficacious in favorably modulating laser induced alterations in vessel diameter and mural connective tissue.

While systemic administration of BAPN-F, i.e. the fumarate salt of Compound VII, to patients for a longer time period is both risky and impractical, the local administration of long acting drug into the wall of the coronary artery has promise. Still, the sustained release of the drug should persist for at least 6 weeks after the angioplasty.

### 2.1.2. Stenosis of the Esophagus

Ingestion of caustic materials is a major management problem which occurs most commonly in children. The development of esophageal stricture is also common following endoscopic sclerotherapy. There are also reports on pill-induced esophageal strictures. The pills are usually tetracycline, potassium chloride, quinidine and others having caustic nature. Esophageal strictures may appear any time after the second week.

Therapeutic management consists of repetitive endoscopic dilations (bougienage) combined with administration of antibiotics and corticoids. This procedure may be continued for up to 18 months after the digestion. Total number of interventions required to achieve adequate dilation varied from 1 to 30, with 14 as a median. In his critical review on caustic ingestion injuries, Kikendal (Gastroenterology clinics of North America 20, 847, 1991) teaches that no therapy has been shown to reduce the severity of the lesions.

Two studies report the beneficial effect of lathyrogens, D-penicillamine (“DPA”) and BAPN, on the esophageal stricture in animals. Butler et al. (Surgery, 81, 431, 1977) inflicted transmural esophageal lye injury in 77 dogs. Twenty-four (24) dogs were untreated controls, twenty-six (26) received corticosteroids and bougienage, and twenty-seven (27) received only BAPN.

The authors concluded that BAPN, given systemically, induced changes in the physical properties of the repair tissue with preventing the stricture of the lumen, all without the need for mechanical dilation by bougienage. In addition the data suggest that wound contracture may play a role in the stricture formation in this animal model.

The other study by Thompson (Laryngoscope, 97, 1191, 1987) induced corrosive esophageal injury in young adult rabbits and treated those with various combinations of colchicine and D-penicillamine. Thompson documented that D-penicillamine alone affected wound healing with less severe stricture and that colchicine was associated with delayed wound healing (due to the inhibition of cellular response to the injury) and severe stricture.

### 1.2. Strictures

### 1.2.1. Urethral Strictures

Every year more than 300,000 patients have transurethral prostatectomy. Of those almost 15 to 30% develop postoperative complications reflecting scar—related strictures. In addition, strictures develop due to gonorrhea resistant infection of the urinary tract. Traumatic etiology such as catheterization or direct mechanical injury to the urethra often results in stricture as well.

The many treatments developed to improve the stricture and its consequences only document that none of these approaches has been satisfactory.
Balloon dilation is effective for a short time, before the ruptured scar tissue regrows. It is therefore not a curative method and in addition has high morbidity;
Temporarily implanted urethral coil stent found more opponents than proponents;
Transurethral resection of the stricture is technically demanding microsurgery, which creates new surgical wounds with possible scarring and strictures;
Urethroplastie in single or multiple steps are technically demanding and costly;
Intralesional corticosteroids injections have questionable effectiveness.

### 1.2.2. Stricture of Sclera Puncture in Glaucoma

An increase of intraoccular pressure is the main reason for various symptoms of glaucoma. Besides conservative treatment, glaucoma surgery developed from the full-thickness procedure to the guarded filtration procedure. Still, some complications persist. Using adjunctive antifibrotic therapy, the success rates increase.

In principle there are two approaches to preserve the patency of the filtration bleb. In a first approach, 5-fluorouracil, mitomycin C, or other cytostatics are used to inhibit the cellular proliferation. Alternatively in a second approach, the scarring and constriction of the sclera puncture is inhibited by preventing collagen crosslinking. Collagen crosslinking was prevented by using lathyrogenic agents, including BAPN-F or DPA.

Roisen et al (Am J. Surg 174,347 1997) inhibited wound contraction after puncturing the sclera with locally injected D-penicillamine and BAPN-F. They also found that collagen deposition in adjacent muscle was decreased. McGuigan et al (Invest Ophthalmol Vis Sci 27, 1755, 1986) found that systemic administration of D-penicillamine was not effective on the function of filter surgery. Moorhead et al (Ann Ophthalmol 19, 223, 1987) used topical BAPN-F in patients after filtration surgery for difficult cases of glaucoma. While the preoperative intraoccular pressure was 40 mm Hg, the postoperative pressure decreased significantly to 19 mm Hg and was maintained for 26.4+−11 weeks. The overall success rate was 74%.

### 1.3. Joint Stiffness

### 1.3.2. Control of Temporomandibular Joint (TMJ) Stiffness and Adhesions by Local Application of BAPN

Ankylosis is defined as a restriction of motion in a joint, resulting most often from a trauma. Injury to the TMJ, to associated muscles and adjacent soft tissue leads to hemorrhaging and inflammation with subsequent fibrotic changes, restricting joint motility. See, Samat and Laskin, The TMJ 1979. Besides trauma and ear infectionimmobilization also results in restriction of normal motion. See, Glineburg et al., The effect of immobilization on primate TMJ, J. Oral Maxillofac. Surg 40, 3, 1982; see also, Lydiat and Davis, The effect of immobilization on the rabbit TMJ, J Oral Maxillofac. Surg. 43, 188, 1985). Lydiat and Davis documented that prolonged immobilization is associated with fibrous adhesions. If left untreated, ankylosis to the TMJ interferes with ingestion, mastication, oral hygiene and speech, thus limiting the quality of life. Ankylosis can occur unilaterally or less commonly bilaterally. When ankylosis occurs during the growth period, it results in varying degrees of facial deformity affecting mainly the lower jaw.

A convenient method to decrease or prevent adhesions would be of significant clinical benefit to patients who have undergone TMJ surgery or having mandibular fracture. The presence of fibrotic adhesions and joint stiffening involving the collagenous structures makes this injury a candidate for treatment with local lathyrogens.

### 1.4. Scar Contractures

Contractures and hypertrophic scars are two of most frustrating sequelae of thermal injury of the skin. The development of hypertrophic scars and the formation of contractures are so common, especially in children, that there are frequently accepted by many physicians as the natural cause of events following thermal injury. (Larson et al 1974).

In the process of wound healing, regardless of the mechanism of injury, there are two events which are often confused. In the early stages of the healing there is a contraction of the wound, where the myofibrils of myofibroblasts provide the energy for centripetal movement of the skin edges. This is, therefore, a cellular phenomenon, which can be inhibited by smooth muscle antagonists, such as Trocinate.

Wound or scar contracture is a later-in-time event related to scar remodeling. This term refers to orientation of collagen fibers within the wound and mainly to their stabilization by intermolecular covalent crosslinks. As indicated above, the result is shrinking of the scar in the form of a contracture or in the healing of tubular organs as stricture or stenosis.

The existing treatment methods for contractures consist of using traction, splinting, and physical rehabilitation, i.e. exercise. While splinting does not affect contraction it is an effective method preventing the scar contracture, if applied for the whole period of lysyl oxidase increased synthesis and activity. Later release of the splint will have a permanent effect on the scar geometry.

Similar considerations apply to the effect of BAPN, which again should be administered throughout the time frame of lysyl oxidase effectiveness. BAPN has never been used for the prevention of scar contractures, either in systemic or topical administration. The study closest to this topic is the research of Richards et al. who found the inhibition of scar contracture of intracardiac prosthetic patches under the treatment by systemic BAPN. (J. Surg. Res. 37, 33, 1984).

### 1.5. Fibrotic Adhesions

### 1.5.1. Peritendinous Adhesions

Trauma resulting in the laceration of the hand tendons, mainly of flexor tendons, or any surgical intervention involving the flexor tendons, bears a risk of forming either an overabundant scar in the healing tendon or forming adhesions between the peritenonium and the peritendinous sheath. Both events result in restriction or complete stop to the gliding of the tendon within the sheath. The process leading to these severe complications is a typical case of fibroproliferative inflammation, involving fibrogenic cells such as typical fibroblasts and also tenoblasts from the peritenonium. The longitudinal alignment of collagen fibers is essential for restoring the mechanical strength of the tendon the collagen fibers.

It was shown that early active mobilization by contraction of the attached muscle is contraindicated as a method promoting the alignment of the collagen fibers, because it stimulates the already excessive deposition of the collagen in the repair tissue and in addition does not improve the tendon gliding (Ketchum, Tendon healing, editors Appleton-Century-Crofts 1979). If, however, at these stages of the healing process a lathyrogen is administered, it prevents the polymerization—maturation—crosslinking of the collagen, which is now more susceptible to degradation by tissue collagenases. It also keeps the collagenous fibrotic adhesions in a fragile form, so that controlled passive mobilization is beneficial. It modifies the quality of the now fragile adhesions.

The risky outcome of hand surgeries involving the flexor tendons actually dissuades some hand surgeons from performing the surgery at all. Once the peritendinous adhesions are formed and stabilized, the tenolysis offers only temporary restoration of the gliding function, as the fibrotic tissue regrows within some 2-3-months.

E. E. Peacock Jr. was the champion of using systemic administration of BAPN in the treatment of peritendinous adhesions in animal models as well in human patients. Based on several animal experiments, he and his coworkers demonstrated a significant improvement in the gliding function of the lacerated tendons in chicken or rats models. See, Peacock and Madden, Surgery 66,215, (1969); see also, Craver, Madden and Peacock, Annals of Surgery 167,697, (1968). Beneficial effects of BAPN in animals were so striking that the same author obtained an IND from FDA to use the drug in patients after tenolysis. Here again he documented a beneficial effect but the dose of BAPN (1-2-g/day per os) was obviously too high inducing several side toxic effects, so that the study was discontinued. Today, with the possibility of local administration of the long acting drug in dosages 100 times lower, BAPN offers new hope to this problem.

### 1.5.2. Perineural Adhesions

There exist certain similarities in the healing of a nerve and a tendon, with the exception that the nerve represents more complex structure and functions. Most of the collagen in the nerve is in the perineurium. This is also the main source of fibrogenic cell invasion into the transected nerve ends or forming the substrate for perineural adhesions. In addition, Swann cells also produce collage, and respond vigorously to nerve injury. Endoneurial fibroblasts multiply as well and lay down collagen around and between the regenerating nerve tubules. Collagen deposition interferes with the nerve budding, as collagen accumulated during reinervation does not resorb as in cutaneous wounds. One of the main deterrents to regaining good nerve function is excessive scar tissue obstructing the properly oriented flow of axoplasm. Thus collagen deposition forms a barrier to axonal regeneration.

In this pathology the main reason for administering BAPN locally is to slow or stop the maturation of collagen into a collagenase-resistant structure. It is known that non-crosslinked collagen is degraded much faster while crosslinked collagen is almost non digestable by the collagenase system.

### 1.6. Potential Candidates for Local Treatment by Long Acting Lathyrogens

### 1.6.1. Dupuytran Contracture

Dupuytran contracture (DC) is a disease of the palmar fascia resulting in the thickening and contracture of the fibrous bands of the hands and fingers. As Liss and Stock indicated a controversy has existed regarding whether acute traumatic injury or cumulative biomechanical work exposure can contribute or initiate this disorder. See, Amer. J. Industr. Med. 29,521 (1996). The authors found good support for the role of vibration in the etiology of DC. DC was reported in rheumatoid arthritis patients, in young children, in the foot.

Bunker and Anthony reported that the pathology of frozen shoulders is reminiscent of the DC pathology with the appearance of thick nodular bands of fibrotic tissue. See, J. Bone @joint Surgery, 77,677 (1995). In this location, the contracture acts as a limitation to external rotation, causing loss of both active and passive movement. The presence of higher amounts of type 3 collagen as well as the incidence of myofibroblasts, increased density and clustering of fibroblasts around the microvessels suggest that the inflammation of the palmar aponeurosis continues to be a factor for final nodules and contractures, limiting the hand movements.

Beneficial continuous extension of DC prior to fasciectomy was reported.

### 1.6.2. Adhesions of Fallopian Tubes

Although the presence of fibrotic adhesions in the tubular tissue seems to be a candidate for the use of locally administered lathyrogens, especially after surgical resection of the blockage, there is minimal or no chance for normal function of the tubes due to the defective action of fimbriae.

### 1.6.3. Carpal Tunnel Syndrome

Carpal tunnel syndrome is a disorder of peripheral neuropathy related to the compression invoked by tendosynovitis in this anatomical region. This disorder originates from various etiologic sources such as occupational injury, acute trauma, repetitive motion, stresses or systemic diseases (diabetes, hypothyroidism, rheumatoid arthritis). The incidence of work related carpal tunnel syndrome has skyrocketed. Once the intracarpal interstitial pressure rises above the critical threshold pressure, capillary blood flow is reduced below the level required for median nerve viability. It is a disorder of middle aged women and middle aged people.

The pathology of tendosynoviyis refers to overproduction of structural macromolecules. Increased collagen degradation is associated with the presence of aggregated collagen, i.e. collagen not yet crosslinked by covalent bonds. And this is the rational for local administration of BAPN-F into the starting lesion.

What is needed is a convenient and effective method to administer one or more lathyrogens at a wound site wherein a single injection of those one or more lathyrogens is effective for a longer time period, thus avoiding the need for daily administration. Applicant's sustained release compositions, comprising one or more lipophilic lathyrongs, and method using those compositions, provides such efficacies.

By “lathyrogen,” Applicant means a substance or combination of substances that produces lathyrism. As those skilled in the art will appreciate, a lathyrogen interferes with the covalent crosslinks in collagen structure which results in the weakening of the mechanical strength of collagen rich tissues. Thus, it leads to bone deformities-osteoporosis, vessel aneurysms, thinning of the skin. The name comes from lathyrus odoratus, sweet pea, containing the effective substance BAPN which was consumed by some cultures and produced the above pathologies.

By “lipophilic,” Applicant means a material having an octanol/water Partition Coefficient (oil/water) greater than about 1. By “hydrophilic,” Applicant means a material having an octanol/water Partition Coefficient less than about 1. As those skilled in the art will appreciate, a Partition Coefficient (oil/water) is a measure of a drug's lipophilicity. That Partition Coefficient is defined as the ratio of un-ionized drug distributed between the organic and aqueous phases at equilibrium.
P_{o/w}=(Cₒᵢₗ/C_{water})_{equilibrium}
The Partition Coefficient is commonly determined using an oil phase of octanol, or chloroform, and water.

For drug delivery, the lipophilic/hydrophilic balance has been shown to be a contributing factor for the rate and extent of drug disposition. Since biological membranes are lipoidal in nature, the rate of drug transfer for passively absorbed drugs is directly related to the lipophilicity of the molecule. Therefore, derivatizing a lathyrogen to increase the Partition Coefficient decreases the rate at which tissue fluids will “wash out” the lipophilic lathyrogen from an injection site, and increases the rate of transfer of that lipohilic lathyrogen through biological membranes.

The following examples are presented to further illustrate to persons skilled in the art how to make and use Applicants' invention, and to identify a presently preferred embodiment thereof. These examples are not intended as limitations, however, upon the scope of the invention, which is defined by the appended claims.

### EXAMPLE I

This example documents one of the effects of DPA on the integrity of collagen structure. It shows that DPA cleaves the non reduced covalent Schiff base crosslinks between the collagen fibrilar molecules and that the effectiveness depends on the presence of both —SH and —NH₂ groups. Once the crosslink is reduced (with sodium borohydrate) a stable covalent crosslinks is formed, resistant to any chemical means to break it. The Example also documents that only substances having —SH and —NH₂ groups free are effective. Therefore N acetyl cystein is not effective due to the blocking its —NH₂ group.

Method: Skin of 4 months old nude mice was dissected, spread on a board and with a 0.5 cm diameter punch several discs were obtained. These were kept in a moist chamber to prevent drying. The discs were weighted and immersed in the following solutions for 1 hour incubation:
1. Phosphate buffered saline (0.1 M, pH 7.3)-control;
2. PBS with 10 mg sodium borohydrate in 10 ml;
3. Some discs from group 1 and 2 were further incubated for 1 hour PBS containing 100 mg cystein, or D-penicillamine or N-acetyl cystein;
4. After 1 hour incubation in the above solutions, the skin samples were placed into 0.45% acetic acid (pH 3.5) for another hour.

After each incubation time the skins samples were blotted on a filter paper and weighted on a analytical balance with the sensitivity to 0.1 mg.

The results are shown in Table 1 and FIG. 1. Swelling in Table 1 is given as mg weight/0.5 cm disc of skin. The data are shown as Mean +/− standard deviation (“SD”).

TABLE 1
SWELLING OF SKIN SAMPLES IN VARIOUS SOLUTIONS

| | Mean | SD |
|---|---|---|
| Control | 36.6 | 3.3 |
| CYSH | 84.0 | 3.5 |
| DPA | 84.0 | 5.0 |
| N-acetylCYSH | 32.2 | 4.5 |
| SBH + control | 27.5 | 5.7 |
| SBH + CYSH | 29.8 | 4.3 |
| SBH + DPA | 26.8 | 5.9 |

The data of Table 1 indicate that only DPA and CYSH were effectively cleaving the double bond in Schiff base crosslinks of the collagen. Once these crosslinks were reduced and the double bond was no more present these drugs were no effective.

In this Example the swelling capacity was used as an indicator of the structural stability, namely number of crosslinks in the collagen molecules. More crosslinked collagen swells less than when the bonds are cleaved, resuming the structure of “young” collagen.

### EXAMPLE II

This example documents that a single injection of lipophilic DPA is effective for an extended period to inhibit the crosslinking of collagen during the healing of a skin wound. Young adult rats have the hair clipped in the dorsum midline to make a rectangle having the dimensions of about 4 cm by about 9 cm. A full thickness skin incision in the midline of the dorsum of the rat is made about 8 cm long. The wound is closed with 9 to 10 interrupted sutures.

Various treatments are injected in between the sutures, underneath the closed wounds with a 8 cm long gastric needle, inserted at the distal site of the incision wound. After 12 days the animals are terminated, the skin wound is dissected with 2 cm intact skin at both sides. A specially designed multiple razor blade cutter with blades 0.5 cm apart is placed onto the skin, stretched loosely onto a cork lined board and hammered to obtain several (10 to 12) equal width skin strips, having the wound in the middle. Individual skin strips are attached to grips of a tensiometer and breaking strength is determined. A breaking strength of such a wound 12 days old, is approximately 300 to 500 g/0.5 cm. depending on the age of the rats.

In previous work, Applicant showed that when the rats are fed a lathyrogen, D-Pencillamine, i.e. Compound I, or beta-aminopropionitrile, Compound II, during the healing period, the tensile strength of the wound is much less, due to the interference of these drugs with the polymerization (crosslinking) of the collagen.

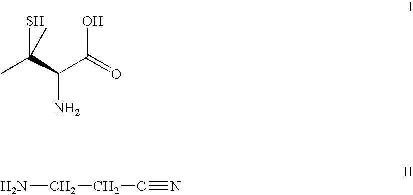

In the Examples II and III, Applicant utilized lipophilic derivatives of Compounds I and II, namely Compounds III and IV, wherein R1 is selected from the group consisting of alkyl, cycloalkyl, phenyl, and benzyl, and wherein R4 is selected from the group consisting of alkyl, cycloalkyl, phenyl, and benzyl.

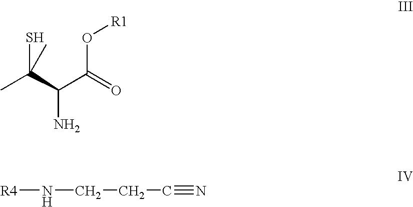

In this Example II, Applicants utilized the above-recited test method to evaluate local administration of unmodified lathyrogens and/or chemically modified lathyrogens, administered in a polymeric carrier.

In certain embodiments, the polymeric carrier comprised a mixture of polylactic and polyglycolic acid of defined molecular weights and proportion, dissolved in N-methylpyrolidone. This polymer was obtained from Atrix Company (CO) as Atrigel 30 which was supposed to be degraded in the tissues within 30 days. When Applicants' formulation mixed with this polymer is injected into body tissues, it solidifies. The rate of its degradation—hydrolysis, determines the rate of the release of the lathyrogen dispersed therein.

The tested lathyrogens, Compound I or derivative thereof, namely D-penicillamine-hexyl ester HCl, i.e. Compound III, wherein R1 is hexyl, was used at the dose of 5 mg/0.25 ml of polymeric carrier (for DPA), or 7.5 mg of Compound III/0.25 ml of polymeric carrier. The formulation was injected after closing the skin wound between the sutures, underneath the wound line. Saline solutions of BAPN and DPA were also administered at the same dose and volume. The results are summarized in the following Table 1.

TABLE II

| | Breaking strength | Change in % of |
|---|---|---|
| Group | (g/0.5 cm) | control |
| 1. Control, non treated | 710 +/− 45 | 0 |
| 2. Control, polymer system only | 655 +/− 65 | −9 |
| 3. BAPN in saline | 735 +/− 40 | +14 |
| 4. DPA in saline | 695 +/− 38 | −2 |
| 5. DPA-hexyl ester. HCl in polymer | 421 +/− 51* | −50 |

Four rats were used in each group. The data recited in Table II are presented as Mean +/− Standard Deviation, based on 20 to 28 determinations in every group. The testing of the breaking strength of the skin wound strips was done 12 days after inflecting the wound. The asterisk appended to the results for Group 5 indicates a statistically significant difference from both controls.

The results show no differences in the breaking strength between non-treated controls and wounds injected with either BAPN and DPA in saline. These results reflect the high solubility and fast wash out these substances from the site of the wound as well as the fast metabolism of tested drugs. In this model of wound healing of incised skin the activity of lysyl oxidase appears 5 days after skin incision and rises to day 12. The enzyme is also quickly resynthetized. The results for Groups 3 and 4 indicate that the free lathyrogens were washed out, at the latest, before day 5.

On the other hand, the results of Group 5, i.e. administration of a modified DPA in the polymeric carrier, shows the effectiveness of the treatment for at least 12 days. If the drug was not available for the enzyme inhibition, the fast resynthesis of lysyl oxidase would normalize the breaking strength of the incision wound. Interestingly, the polymeric carrier was still present underneath the wound and was encapsulated by loose connective tissue.

Moreover, the formulation containing the modified DPA as hexyl ester dispersed in the polymeric carrier was effective in spite the fact that possibly only less than about half of the dose of DPA was released from the carrier. Most of the polymer was found in the wound at time of termination. This finding indicates the effectiveness of the modified DPA to inhibit the formation of crosslinks in collagen.

### EXAMPLE III

Example III shows the effectiveness of hexyl-beta-aminopropionitrile, i.e. Compound IV wherein R4 is hexyl, and no effect of lipophilic DPA-hexanal acetone adduct. Both drugs were mixed in polyethylene glycol 600 to form a homogenous emulsion.

The experimental conditions were the same as in Example II. The dose for hexyl(amino)propionitrile and for DPA-acetone adduct was 9 mg/0.25 ml of the polymer. As the PEG 600 is water soluble, the modified lipophilic drugs were emulsified in the polymer by vigorous mixing at 37° C. keeping the polymer fluid.

The data of Table III are based on 16 to 22 determinations in each group. Variability is given as SD.

TABLE III

| | X | SEM |
|---|---|---|
| CONTROL | 479 | 17 |
| POLYMER-PEG-600 | 480 | 14 |
| BAPN - hexyl amide in PEG | 264* | 21 |
| DPA - acetone adduct in PEG | 499 | 27 |

The interpretation of these results requires some information. It is known that in order for BAPN to be an irreversible inhibitor of L.O the free amino group is essential In our case one hydrogen of the NH2 group is linked to the amide. Still, it was documented that even the imides of BAPN are inhibitory to L.O., as also in indicated by this Example.

In Example I we documented that DPA is inhibiting L.O. activity only when both SH and NH2 in adjacent positions are available. But in this experiment we blocked both groups by forming a ring called acetone adduct, which is a form of Schiff base. It is known that this link is cleaved by thiols. We assumed that in the body the ubiquitous presence of glutathione or cystein will continuously cleave this ring and free both functional groups allowing the interaction with L.O. This assumption proved wrong and as shown in Example III there is no effect of this lipophilic compound on the breaking strength (crosslinks) of the healing wound.

The hydrophilic polymer was not present in histologically or macroscopically analyzed harvested wounds. It can be concluded that lipophilic BAPN in PEG was effective to block L.O. for at least 12 days.

### EXAMPLE IV

This example documents the effectiveness of a single injection of both lipophilic DPA and BAPN. This example uses the same animal model of wound healing of incision skin wound in rats. The progress of healing was measured by determining the breaking strength of them wound 12 days after locally infiltrating the sutured wound underneath of the skin wound with lipophilic lathyrogens, hexyl ester-DPA.HCl and hexyl amide (amino)propionitrile. The method was same as in previous Examples II and III. There were 4 Sprague-Dawley female rats, 200-210 gram body weight, in every group. Twelve days after inflicting the wound and injecting the drugs in thermosensitive polymer (TSP-kindly provided by Dr A. Gutowska, Battelle, Wash.) the rats were terminated, skin wound dissected, spread evenly on a cork board and 0.5 cm strips of the wound cut off using a tool with serial blades, 0.5 cm apart. The strips of the skin, 18-24/group of 4 rats, were tested in a Instron tensiometer. The results are summarized in Table IV.

TABLE IV
Breaking strength of skin wound.
(Data shown as X +/− SD)

| | | |
|---|---|---|
| Control | 481 | 49 |
| TSP only | 421 | 77 |
| BAPN - lipophilic | 226 | 57 |
| DPA - lipophilic | 321 | 43 |

We found a highly significant reduction of the breaking strength-related to the inhibition of the formation or cleavage of existing labile Schiff base covalent crosslinks in the collagen structure of the healing wound. The polymer used in this study (TSP) had no effect on the healing and properties of collagen.

This study documents that Applicant's lipophilic lathyrogens in a single local injection into the site of the injury are effective inhibitors of collagen stabilization by the formation of covalent intermolecular crosslinks. In certain embodiments, DPA-derived lipophilic lathyrogens, including for example the methyl, hexyl, and benzyl esters, are prepared as hydrochloride salts. Applicant has found that these salts in solution show enhanced stability, dissolution rate, and resistance to oxidation. Subsequent to injection into a tissue structure and neutralization by tissue fluids, the resulting lipophilic lathyrogen is retained within the injected tissue.

While the preferred embodiments of the present invention have been illustrated in detail, it should be apparent that modifications and adaptations to those embodiments may occur to one skilled in the art without departing from the scope of the present invention as set forth in the following claims.

1. A method to inhibit crosslinking of collagen during the healing of a wound, comprising the steps of:
providing a lipophilic lathyrogen;
providing a patient having a wound in a tissue structure, wherein said wound is defined by a wound line;
injecting said lipophilic lathyrogen into said tissue structure adjacent said wound line.

2. The method of claim 1, wherein said lipophilic lathyrogen comprises a compound having the structure 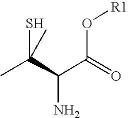
wherein R1 is selected from the group consisting of alkyl, cycloalkyl, phenyl, and benzyl.

3. The method of claim 2, wherein R1 is hexyl.

4. The method of claim 3, wherein said lipophilic lathyrogen is mixed with a polymeric carrier.

5. The method of claim 4, wherein said polymeric carrier comprises polylactic acid and polyglycolic acid dissolved in N-Methylpyrrolidone.

6. The method of claim 5, wherein said lathyrogen is used at a dose of 5 mg per 0.25 mL of polymeric carrier.

7. The method of claim 1, wherein said lipophilic lathyrogen comprises a compound having the structure 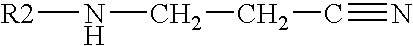
wherein R2 is selected from the group consisting of alkyl, cycloalkyl, phenyl, and benzyl.

8. The method of claim 7, wherein R2 is hexyl.

9. The method of claim 8, wherein said lathyrogen is mixed with a polymeric carrier.

10. The method of claim 9, wherein said polymeric carrier comprises polyethylene glycol having a molecular weight of about 600 Daltons.

11. The method of claim 10, wherein said lathyrogen is used at a dose of 9 mg per 0.25 mL of said polymeric carrier.

12. A sustained release composition effective for inhibiting crosslinking of collagen during wound healing, comprising a lipophilic lathyrogen.

13. The composition of claim 12, wherein said lipophilic lathyrogen comprises a compound having the structure 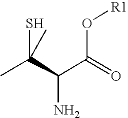
wherein R1 is selected from the group consisting of alkyl, cycloalkyl, phenyl, and benzyl.

14. The composition of claim 13, wherein R1 is hexyl.

15. The composition of claim 14, further comprising a polymeric carrier.

16. The composition of claim 15, wherein said polymeric carrier comprises polylactic acid and polyglycolic acid dissolved in N-Methylpyrrolidone.

17. The composition of claim 16, wherein said composition comprises 5 mg of said lipophilic lathyrogen per 0.25 mL of said polymeric carrier.

18. The composition of claim 12, wherein said lipophilic lathyrogen comprises a compound having the structure 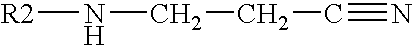
wherein R2 is selected from the group consisting of alkyl, cycloalkyl, phenyl, and benzyl.

19. The composition of claim 18, wherein R2 is hexyl.

20. The composition of claim 19, further comprising a polymeric carrier.

21. The composition of claim 20, wherein said polymeric carrier comprises polyethylene glycol having a molecular weight of about 600 Daltons.

22. The method of claim 10, wherein composition comprises about 9 mg of said lathyrogen per 0.25 mL of said polymeric carrier.
